(19) United States
(12) Patent Application Publication
(10) Pub. No.: US 2026/0298876 A1
(43) Pub. Date: Oct. 1, 2026

(54) Measuring device and a measuring method for determining parameters of flat hygiene paper and textile-like or textile sheet materials

(71) Applicant: emtec Electronic GmbH, Leipzig (DE)

(72) Inventor: Giselher Gruener, Leipzig (DE)

(21) Appl. No.: 19/489,833

(22) PCT Filed: May 30, 2024
(86) PCT No.: PCT/EP2024/064879

§ 371 Date: Dec. 3, 2025

(30) Foreign Application Priority Data
Jun. 5, 2023 (DE) 10 2023 114 690.4

(51) Int. Cl. G01N 29/04; G01N 29/12; G01N 29/22; G01N 29/44; G01N 33/34; G01N 33/36; G06T 7/00

(52) U.S. Cl. CPC G01N 29/045; G01N 29/12; G01N 29/225; G01N 29/4472; G01N 33/346; G01N 33/36; G06T 7/0004; G01N 2291/014; G01N 2291/0237; G06T 2207/30124

(57) ABSTRACT

The invention relates to a measuring device (100, 100′) for determining individual parameters of sheet materials, such as flat hygiene paper and flat, textile-like or textile materials. Methods are described for determining the individual parameters by means of the measuring device (100, 100′).

### CROSS-REFERENCE TO RELATED APPLICATIONS

This Application is a § 371 national phase entry of International Patent Application No. PCT/EP2024/064879 filed on May 30, 2024, which claims the benefit and priority to German Application 102023114690.4 filed on Jun. 5, 2023. The entire contents of these applications are incorporated herein by reference in their entireties.

The invention relates to a measuring device and a measuring method for determining parameters of flat hygiene paper and textile-like or textile sheet materials.

Publication WO 2007/093484 A1 discloses a method and a measuring device for determining the softness of a sheet material, in particular flat hygiene paper and flat textile materials. The measuring device comprises an element which is arranged to be movable relative to a positionally fixed, at least single-layer sample of the sheet material and which is configured to be adjustable relative to the sample according to a predetermined pressing force acting on the sample, wherein a microphone is arranged in the region of the vibrations that occur between the sample and the element, which microphone records the noises produced by the relative movement of the element acting on the sample. The associated method is characterized in that the element, which is movable relative to the positionally fixed, at least single-layer sample of sheet material and which acts on the sample with a predeterminable pressing force, produces a noise in a predeterminable measuring period, which is received and recorded, wherein the vibrations generated are detected and evaluated by means of vibration analysis by determining a sound spectrum or a frequency band, and a specific softness of the sample of sheet material is assigned to each determined sound spectrum or frequency band.

The prior art also includes the publications U.S. Pat. Nos. 3,383,681 A and 3,060,719 A.

Based on publication DE 10 2006 007 678 A1, it has become apparent that determining the softness of a sample of sheet material is still considered an important or even the most important parameter, but users would now like additional information on certain parameters of said sheet materials.

The background to this is simply that users are beginning to look for additional individual parameters as quality parameters of sheet materials, which fall under the umbrella term of ‘haptics’, wherein the umbrella term ‘haptics’ is now also regarded as an overall quality parameter.

Manual haptic quality testing by the user involves touching the sheet material to be tested with a human hand. This means that there is an interaction between the material and certain sensors in the fingers of the person performing the test, which are stimulated by the texture of the fingertips and the papillary lines. The sensory signals produced in this way are transmitted to the brain, where they are processed—in different ways for each user—into a subjective impression of ‘haptic quality’.

The haptic sensation or ‘haptic quality’ is therefore a subjective quality parameter that depends on the user and consists of a combination of different parameters. These parameters are various individual properties that are initially relatively independent but ultimately work together to shape the haptic sensation.

The objective of the invention is to determine at least one additional parameter for sheet materials, in addition to the known parameter of softness, which will help users to compare at least one or more previously unavailable properties of a sheet material or several different sheet materials, which describe the respective sheet material itself or enable an objective comparison to be made of supposedly similar or different sheet materials with regard to these properties.

The starting point of the invention is a measuring device for determining individual parameters of sheet materials, such as flat hygiene paper and flat, textile-like or textile materials, wherein the measuring device comprises a measuring head and a measuring housing, wherein a scraper element is arranged on the measuring head which element is movable relative to a positionally fixed, at least single-layer sample of the sheet material arranged on the measuring housing and is arranged to be adjustable relative to the sample according to a predetermined pressing force acting on the sample.

According to the invention, a first microphone is arranged inside the measuring housing, which forms in its interior an enclosed measuring chamber underneath the sample, and a second microphone is arranged outside the measuring housing, which together or independently of each other record noises produced by the relative movement of the scraper element acting on the sample.

It is preferred that at least one vibration generator is arranged in the measuring head.

Furthermore, it is preferably provided that the measuring head comprises a first motor, a second motor and a third motor, wherein the first motor causes a rotational movement of the scraper element relative to a sample surface of the positionally fixed, at least single-layer sample, while the second motor causes an orthogonal translational movement or a substantially orthogonal translational pendulum movement of the scraper element relative to the sample surface of the positionally fixed, at least single-layer sample, and the third motor causes an orthogonal displacement of a temperature generation and measuring device movably arranged on the measuring head relative to the sample surface of the positionally fixed, at least single-layer sample.

In a further preferred embodiment, the first motor of the measuring device is connected to a measuring device which determines and records a current curve and/or a torque and/or power consumption of the first motor during the rotational movement of the scraper element generated by the first motor.

In yet another preferred embodiment, at least one temperature sensor is arranged in the measuring chamber at a predeterminable distance from the positionally fixed, at least single-layer sample.

Finally, in a further preferred embodiment, at least one camera is arranged in or on the measuring head.

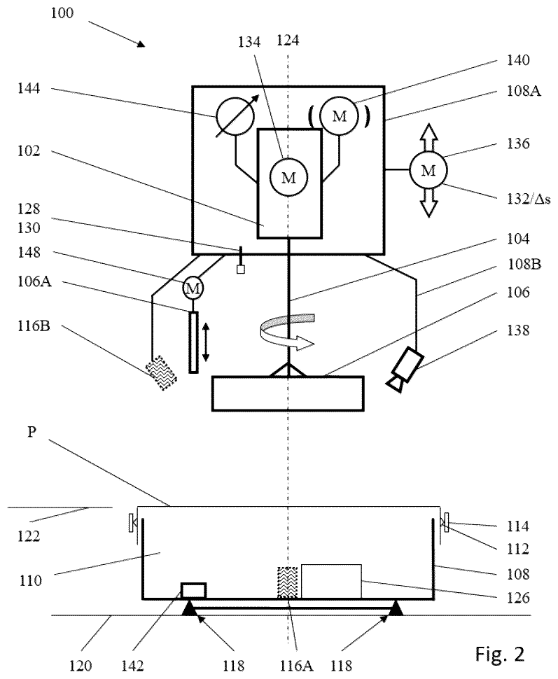

According to the preferred embodiments described above, the measuring device may have only some of the said preferred features or may have all of them. However, preferred features can also be omitted in some embodiments. FIGS. 2 to 6 show the measuring device in which all of the preferred features are implemented so that all subsequent method steps of the invention can be carried out.

The measuring device makes it possible to carry out a series of method steps, which are performed as follows according to the following information:

The measuring device makes it possible to carry out a method for determining individual parameters of sheet materials, such as flat hygiene paper and flat, textile-like or textile materials, wherein in a separate method step, a movable scraper element is moved in translation relative to a positionally fixed, at least single-layer sample of the sheet material onto the sample with a predeterminable pressing force, while simultaneously rotating the movable scraper element, and makes contact with and acts on said sample, thereby generating, receiving and recording a noise in a predetermined measuring period.

According to the invention, it is provided that the vibrations generated are detected by means of vibration analysis to determine a sound spectrum or a frequency band with a first microphone arranged inside a measuring housing which forms in its interior an enclosed measuring chamber underneath the sample, and/or a second microphone arranged outside the measuring housing, and then evaluated, wherein a specific softness and a specific roughness of the sample are assigned to the determined sound spectrum or frequency band as individual parameters based on specific peaks in the sound spectrum or frequency band, wherein a specific softness and a specific roughness are assigned to the sheet material as individual parameters of the sample of the sheet material based on the specific peaks determined in the sound spectrum or frequency band by applying a calculation algorithm.

The measuring device also allows a method to be carried out for determining individual parameters of sheet materials, such as flat hygiene paper and flat, textile-like or textile materials, wherein, in a separate method step, the movable scraper element is moved exclusively in translation relative to a positionally fixed, at least single-layer sample of the sheet material onto the sample with a predeterminable pressing force and makes contact with and acts on said sample, whereby noise is generated, received and recorded in a predetermined measurement period.

This method step is characterized in that, during the predetermined measurement period, a vibration generator causes the movable scraper element to vibrate, wherein the vibrations produced on the sample side are detected by means of an additional vibration analysis to determine an additional sound spectrum or frequency band, which is recorded by the first microphone arranged inside the measuring housing and then evaluated. The measuring housing forms in its interior an enclosed measuring chamber underneath the sample. Within the determined additional sound spectrum or frequency band, a peak is generated and recorded as a single parameter at a vibration frequency that can be specified by the vibration generator in the sound spectrum, and the significance of this is explained in the following.

It was found that the vibration of the sample produced by the vibration generator—now rotation-free—in the additional sound spectrum or frequency band also forms a peak with a specific height and within the additional sound spectrum or frequency band in a specific position, wherein it was also found that the position and height of the peak depends on the respective surface weight and the respective porosity of the sheet material being examined. The specific peak determined and its position in the additional sound spectrum or frequency band is advantageously a variable the measure of which characterizes the porosity of the sheet material, wherein the variable is also determined using a calculation algorithm. Since the peak in the measuring housing is formed by the vibration of the sample in the additional sound spectrum or frequency band, which is generated without rotation by the vibration generator, this peak is referred to as the NRV peak, or non-rotational vibration peak, of the sample.

According to the invention, it is provided that the previously described method step for assessing softness and roughness—without the vibration generator—is carried out before or after the previously described method step with the vibration generator—to determine the NRV peak produced by the vibration generator, wherein it is advantageously provided that the specific peaks of the individual parameters of softness and/or roughness determined in the sound spectrum or frequency band are transformed by means of a mathematical correction function. This mathematical correction function transforms the specific peaks of the individual parameters of softness and/or roughness determined in the sound spectrum or frequency band as a function of the position and height of the peak or peaks formed by the rotation-free vibration produced on the sample side in the additional sound spectrum or frequency band, so that the dependency of the softness and/or roughness of the sheet material on the porosity of the corresponding sheet material can now be taken into account in an advantageous manner by the peak characterizing the porosity. This means that by combining the results of the method steps, an individual parameter of porosity-corrected softness and/or an individual parameter of porosity-corrected roughness is/are advantageously obtained. In other words, the correction function is applied which transforms and corrects the influence of porosity with regard to the specific peaks of the individual parameters of softness and/or roughness by applying the correction function within the calculation algorithm for determining softness and/or roughness, which takes into account the position and height of the previously explained NRV peak in the additional sound spectrum or frequency band.

The measuring device also makes it possible to carry out a method for determining individual parameters of sheet materials, such as flat hygiene paper and flat, textile-like or textile materials, wherein the movable scraper element is moved in translation onto the sample in a further separate method step relative to a positionally fixed, at least single-layer sample of the sheet material with a predeterminable pressing force, while simultaneously rotating the scraper element, and makes contact with and acts on said sample, wherein here according to the invention during the generated rotational movement of the scraper element in a predetermined measuring period, a current curve and/or or a torque and/or power consumption of a first motor, which causes the rotational movement, is/are determined and recorded, so that, depending on the respective measurement result, a specific frictional force is indirectly derived as an individual parameter and is assigned to the sample, which frictional force is generated by the contact between the sample and the scraper element during the rotational movement.

The measuring device also makes it possible to carry out a method for determining individual parameters of sheet materials, such as flat hygiene paper and flat, textile-like or textile materials, wherein, according to the invention also in a separate method step, a movable temperature generation and measuring device is moved in translation relative to a positionally fixed, at least single-layer sample of the sheet material with a predeterminable pressing force onto the sample and makes contact with and acts on a surface of said sample, wherein the sheet material is either heated to a specific predetermined temperature by means of the temperature generation and measuring device in a predetermined measurement period and the temperature change of the sample per unit of time is simultaneously recorded in the predetermined measurement period, or a specific heating current is applied to the temperature generation and measuring device for a predeterminable time, after which the temperature curve of the movable temperature generation and measuring device is determined during this time, so that, depending on the measurement results, a specific surface thermal conductivity is derived as a single parameter and assigned to the sample.

The measuring device also makes it possible to carry out a method for determining individual parameters of sheet materials, such as flat hygiene paper and flat, textile-like or textile materials, wherein, according to the invention in a further separate method step, a movable temperature generation and measuring device is moved in translation relative to a positionally fixed, at least single-layer sample of the sheet material with a predeterminable pressing force onto the sample and makes contact with and acts on a surface of said sample, wherein the sheet material is heated to a specific predetermined temperature by means of the temperature generation and measuring device in a predetermined measurement period, wherein furthermore underneath the sample, on a side of the sample opposite the temperature generation and measuring device, at a predetermined distance from the sample, a temperature sensor is arranged which detects a temperature, wherein the detected temperature is determined and recorded in a characteristic curve over time, so that, depending on the measurement results, a specific thermal insulation capacity is derived as an individual parameter and assigned to the sample.

In a further advantageous manner, the measuring device can also be used to carry out a method for determining individual parameters of sheet materials, such as flat hygiene paper and flat, textile-like or textile materials, wherein the movable scraper element is moved in translation, in a further separate method step, relative to a positionally fixed, at least single-layer sample of the sheet material with a predeterminable pressing force onto the sample and makes contact with and acts on said sample, which adopts a deflected deformation position, wherein, according to the invention, it is provided that, in a predetermined measurement period, a displacement differential covered by the scraper element when pressing the scraper element onto the sample is determined and recorded, so that, depending on the measurement results, a certain lateral flexibility is derived as an individual parameter and assigned to the sample.

Finally, the measuring device is also used to carry out a method for determining individual parameters of sheet materials, such as flat hygiene paper and flat, textile-like or textile materials, wherein the movable scraper element is moved in translation, in a separate method step, relative to a positionally fixed, single-layer sample of the sheet material, or the movable scraper element is moved in translation, also in a separate method step, relative to a positionally fixed, multi-layer sample of the sheet material with a predeterminable pressing force onto the sample and makes contact with and acts on said sample, wherein, according to the invention, it is provided that the pressing force is a predetermined test force which moves the sample from its rest position into a deflected deformation position in at least one measurement cycle, wherein, in the at least one measurement cycle, a speed at which the predetermined test force is to be reached is specified, wherein, in the at least one measurement cycle, the sample returns to its final deformation position or its original rest position at a predetermined relief time point, at which the test force is withdrawn, wherein, in a predetermined measurement period of the measurement cycle, a displacement differential covered by the sample and a time period in which the sample returns from the deflected deformation position to the final deformation position or the original rest position are determined and recorded, so that, depending on the measurement results, a resilience of the sample is derived as an individual parameter and assigned to the sample, wherein, furthermore, a number of measurement cycles and/or the speed at which the sample is deformed and/or the relief time point at which the test force is withdrawn and/or the test force at which the resilience is determined can be specified.

It is also preferably provided that, in the method for determining individual parameters of sheet materials, such as flat hygiene paper and flat, textile-like or textile materials, a further separate method step can be carried out by the measuring device, in which the surface structure of the sample is photographed by a high-resolution camera, so that visual image documentation of the sample is available as an individual parameter, wherein further individual parameters are verified as a function of the photographic reproduction result of the surface structure.

The invention is explained in more detail in the following. In the Figures:

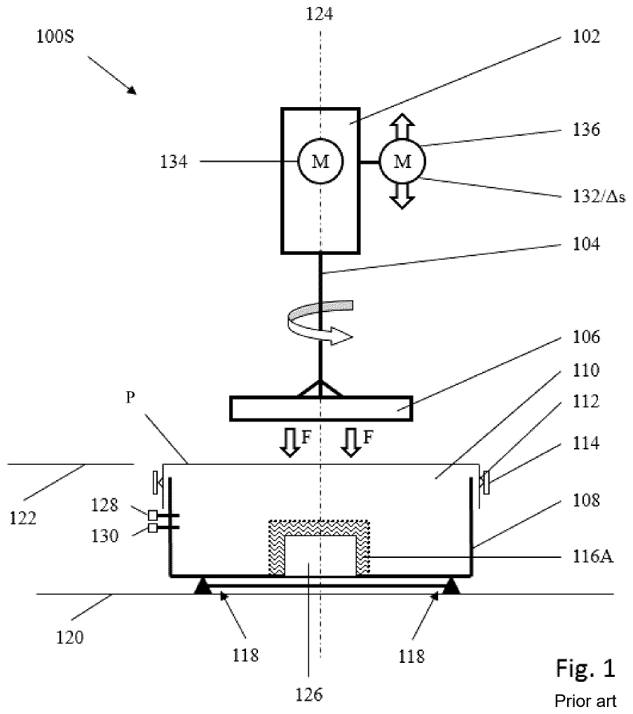

FIG. 1 shows a conventional measuring device for determining the softness of the sheet material in an open state;

FIG. 2 shows a measuring device according to the invention in a first embodiment in an open state;

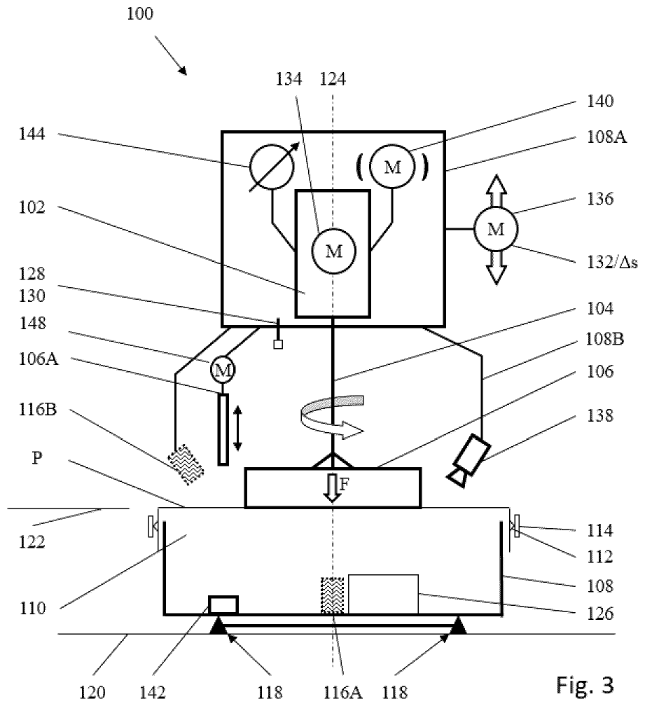

FIG. 3 shows the measuring device according to the invention as shown in FIG. 2, now in a closed operating state in an application state for explaining the detection of individual parameters, namely the softness and roughness of the sheet material;

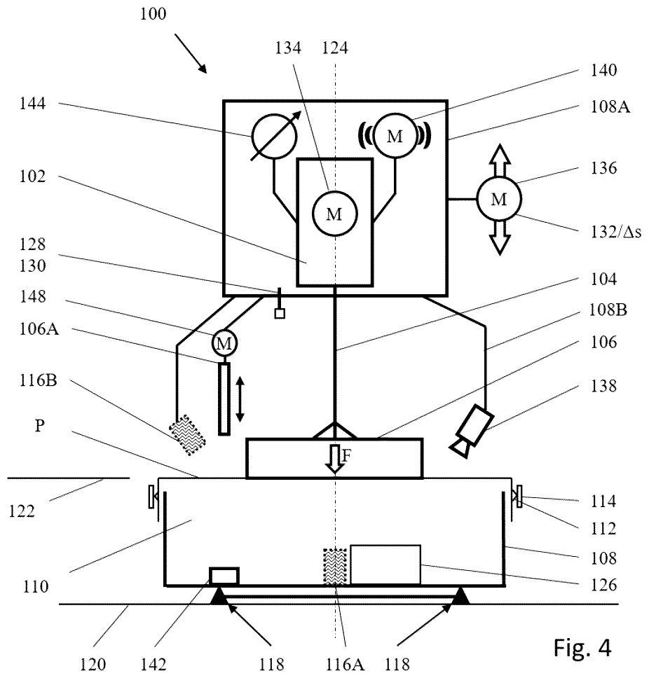

FIG. 4 shows the measuring device according to the invention as shown in FIG. 2, now in a closed operating state in another application state for explaining the detection of a further variable characterizing the sheet material, from which a porosity-corrected softness and/or a porosity-corrected roughness of the sheet material can be derived as individual parameters;

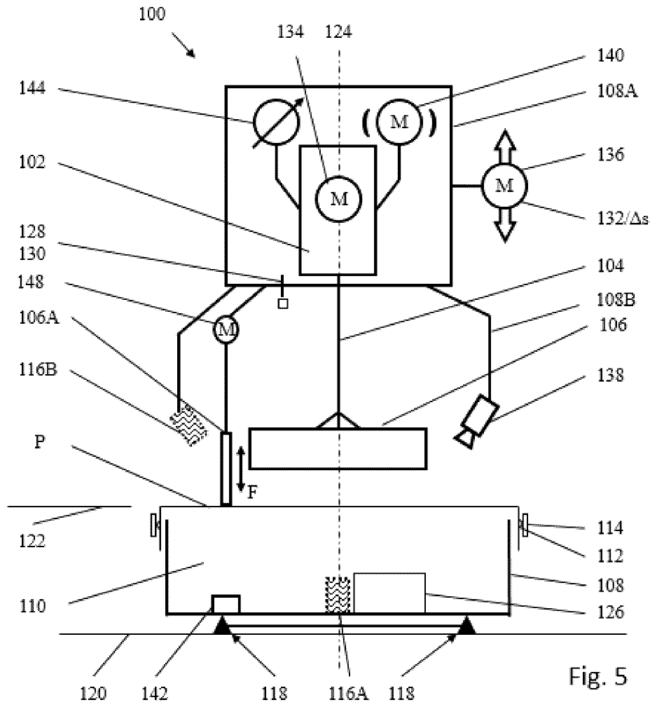

FIG. 5 shows the measuring device according to the invention as shown in FIG. 2 in the closed operating state in another application state for explaining the detection of further individual parameters, namely a surface thermal conductivity and a thermal insulation of the sheet material;

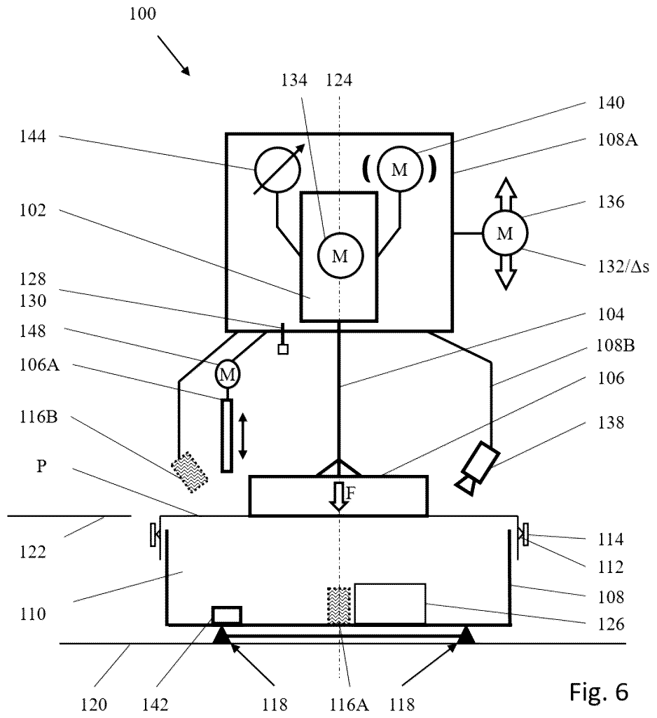

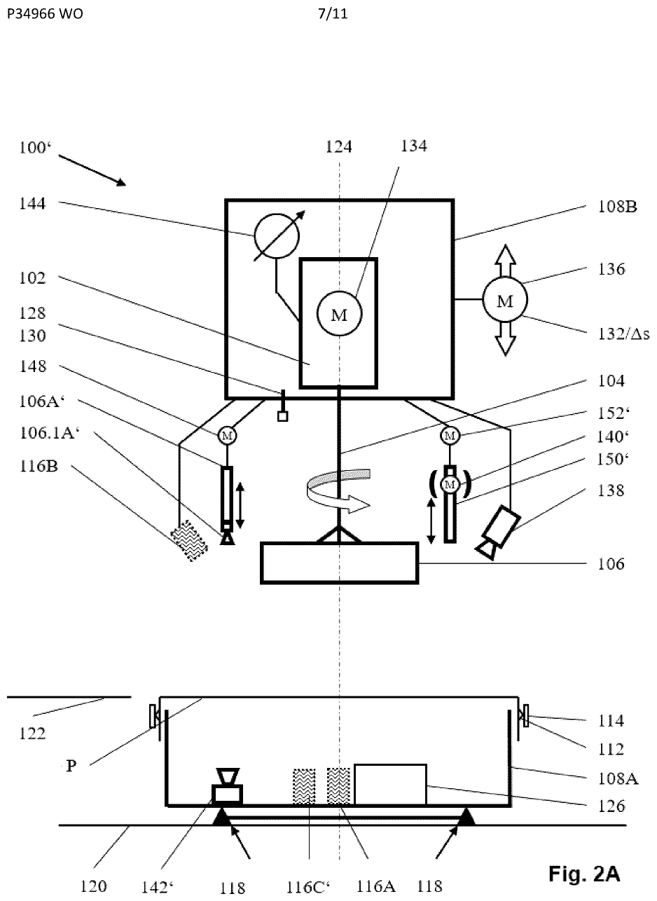

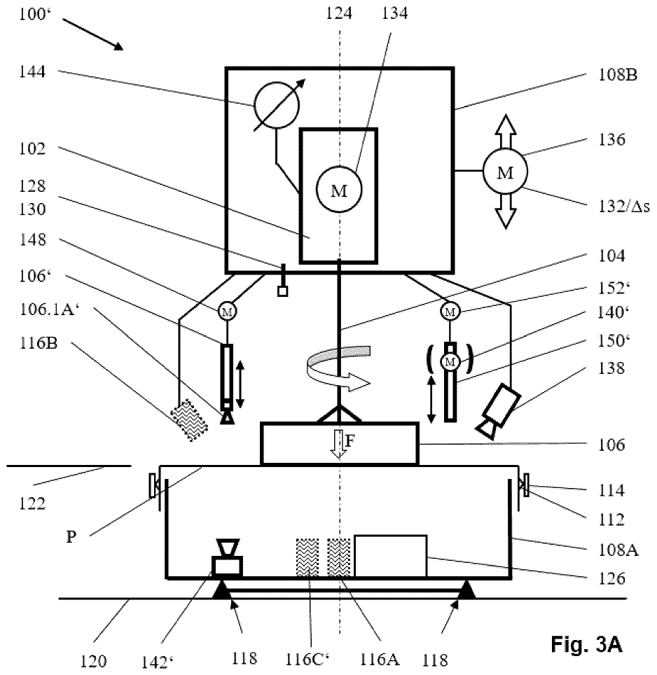

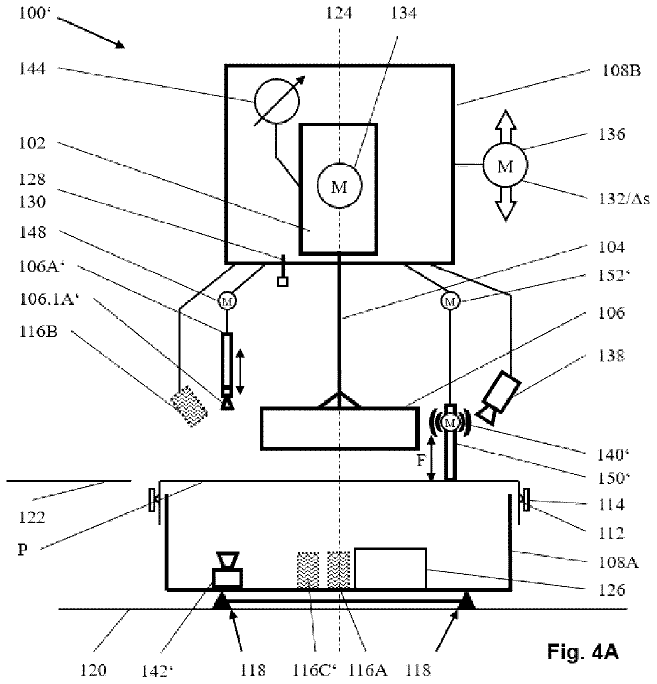

FIG. 6 shows the measuring device according to the invention as shown in FIG. 2, now in the closed operating state in another application state for explaining the detection of further individual parameters, namely a lateral flexibility of the sheet material and a resilience of a single-layer sheet material and a resilience of a multi-layer sheet material.

According to FIG. 1, the measuring device 100S known from the prior art comprises a movable element, which is referred to in the following as a scraper element 106.

FIG. 1 shows the measuring device 100S in an open state, in which the scraper element 106 does not lie on a sample P. This scraper element 106 is connected via a transmission means 104—in the embodiment, a drive shaft—to a drive 102, which causes the scraper element 106 to rotate on an axis 124 via a first motor 134, which is shown schematically in the drive 102. The drive 102 is provided with a second motor 136, which is shown next to the drive 102, so that the scraper element 106 can perform a translational movement along the axis 124 in addition to the rotational movement about the axis 124. This translational movement is performed until a predeterminable pressing force F is achieved, which is measured by a force measuring device 118.

In parallel to this, the displacement differential Δs is measured by a displacement measuring device 132, from which an individual parameter can be derived that corresponds to the elasticity of the sample P of the hygiene paper or textile.

In the present embodiment, the scraper element is rotatable relative to the sample P and adjustable, in particular height-adjustable relative to the sample P. The adjustment here is therefore vertical to the horizontally arranged sample P.

The invention is not limited to a rotary movement relative to the sample P, but rather also includes translational movements or pendulum movements that the scraper element 106 can perform relative to the sample P, wherein these movements performed relative to the sample P are coupled. With regard to the predeterminable displacement differential Δs, that the scraper element 106 can perform relative to the sample P starting from an initial position, the predetermined translational adjustment movement makes it possible to specify a pressing force F on the sample P. This pressing force F, which acts on the sample P, and the example of its direction of action are shown in FIG. 1 by means of arrows pointing in the direction of the sample P. A direction of action of the pressing force F is also possible which does not act vertically to the horizontal sample P, but which acts at a predeterminable angle different from 90°.

The scraper element 106 is assigned a measuring housing 108, which is constructed from vertical side walls and a horizontal base wall. Such a measuring housing 108 is not absolutely necessary for carrying out the previous method. In principle, the measuring device 100 only needs to have a holding device to which or in which the sample P can be attached.

In the preferred embodiment, the side walls of the measuring housing 108 are used to secure the sample P, on which the sample P of the respective test material (sheet material) is placed or clamped, for example, by simply folding over the edge areas of the sample P, wherein the attachment is preferably carried out via a sealing element 112, in particular a rubber lip, and by means of a holding element 114, preferably a clamp, a rubber band or the like. This arrangement results in a sample plane 122 arranged above a measuring chamber 110 in the measuring housing 108. As already mentioned, such a measuring chamber 110 is not absolutely necessary in the conventional measuring device 100S.

The arrangement of the sample plane 120 could also be formed above an open region without corresponding side walls or base walls of the measuring housing 108 without forming an enclosed measuring chamber 110.

However, it is preferable to provide such a measuring housing 108 forming a measuring chamber 110 in which a temperature sensor 128 and a humidity sensor 130 is arranged for measuring a temperature T or a relative humidity ω (cf. FIGS. 2 to 6 above, with a humidity and temperature sensor 128/130 arranged as a combination element).

The arrangement is preferably in one of the side walls of the measuring housing 108, although it can of course also be arranged independently of the measuring housing 108 if corresponding side walls are not formed.

Overall, it should be mentioned that the measuring device 100S can also be arranged in a climate room or climate chamber in which constant temperature conditions or relative humidity conditions ω prevail, wherein, preferably for standardization of the measurement, the normal conditions, a temperature T of T=23° C. and a relative humidity of ω=50% are used as standard data, which are also generally known from the prior art.

A first microphone 116A is arranged inside the measuring housing 108 or in the region of a holding device and is connected to an evaluation and calibration unit 126.

In the known embodiment, the evaluation and calibration unit 126 is arranged in the first microphone 116A.

However, these components 116A, 126 can also be configured separately, so that the arrangement shown is only given as an example.

FIG. 1 shows that the measuring housing 108 is arranged on the force measuring device 118, which is fixed relative to a base 120 and by means of which the pressing force F acting directly on the sample P via the scraper element 106 is measured indirectly.

The first microphone 116A and also the second microphone 116B explained in the following are examples of any type of measuring device capable of receiving and recording sound spectra or frequency bands, since the known method involves performing a vibration analysis of the noises produced between the scraper element 106 and the sample P.

The previous method is explained in more detail with reference to FIG. 1 as the starting point of the invention.

The method for determining the softness of hygiene paper is carried out as follows:

A corresponding sample P is taken from a batch of hygiene paper or textile available in the practice and clamped onto the measuring housing 108 by the holding element 114.

The height-adjustable scraper element 106 is first applied to the flexible sample P via the drive 102 using the second motor 136 with a predeterminable pressing force F. A preferably material-dependent adjustable pressing force F is, for example, 0.1 N, wherein an adjustment distance Δs is also specified and then measured and monitored. The adjustable pressing force F is also selected in such a way that the measurement results explained later with regard to the softness and roughness correlate with a manually performed examination of the sheet material.

The scraper element 106 is set in rotation by the drive 102 by means of the first motor 134 and produces a noise for a predeterminable time period Δt which propagates in waves, generating complex vibrations in the region of the sample P.

A preferred rotational speed is approximately between 1 Hz and 3 Hz, although speeds above and below this range may also be used. The adjustable pressing force F is selected such that the sample P does not tear.

The method can also be designed such that the adjustment movement Δs and the rotational movement begin simultaneously, so that a noise is produced in that the scraper element 106 is pressed onto the sample P with the pressing force F during its rotation.

These vibrations, which are within or outside the human hearing range, form the basis for the aforementioned vibration analysis and for determining sound spectra or frequency bands, wherein the term ‘sound’ is also used in the following for vibrations that are both within and outside the human hearing range.

The vibrations are recorded by the first microphone 116A, which is arranged in the measuring chamber 110 according to FIG. 1, in which a corresponding sound field is formed.

The arrangement of two microphones 116A, 116B is used according to the invention to determine a parameter that has not yet been determined, as explained in the following.

It should be noted once again that, in order to ensure the comparability of several measurements, a measuring chamber 110 with defined conditions, such as relative humidity ω=50% and temperature T=23° C., and thus constant environmental conditions (without background noise, etc.), is preferable. For textiles, a different standard is applied for the measurements, according to which a relative humidity ω=65% and a temperature T=20° C. are set and monitored as constant environmental conditions.

The evaluation and calibration unit 126 belongs to a control device, wherein the evaluation and calibration unit 126 performs an evaluation of recorded vibrations by means of vibration analysis, wherein preferably a sound intensity I and/or a sound level Lp of the sound pressure p is evaluated and calibrated in predeterminable ranges of the sound spectra and/or frequencies of the frequency bands.

The control device also ensures the described positioning movements of the scraper element 106 relative to the positionally fixed sample P or vice versa, depending on the embodiment used.

The control device can control and regulate the rotational/translational pendulum movement of the scraper element 106 and the adjustment movement Δs of the sample P or vice versa and the time period Δt provided for this, whereby the preset pressing force F can also be controlled or regulated via the control device.

The further adjustment movements and evaluations according to the invention explained in the following are additionally integrated into the control device.

### Invention:

It has been found that, in addition to softness, other individual parameters of sheet materials are also of interest to users, which can now be detected or assessed using a measuring device 100 according to the invention.

The new, further developed measuring device 100 and the new methods according to the invention for determining or assessing further individual parameters of the sheet material are explained in the following with reference to the schematic representations in FIGS. 2 to 6. Identical components are always denoted by the same reference signs.

The following materials are defined in the following as sheet materials that can be examined by the measuring device 100 according to the invention or on which the methods explained in the following can be performed.

Firstly: “Tissue/hygiene paper”; absorbent, finely creped hygiene paper made from cellulose pulp. Single-ply or multi-ply as toilet paper, paper tissues, but also kitchen paper, paper serviettes.

Secondly: “Nonwoven”; structures made of fibers of limited length, continuous fibers (filaments) or cut yarns of any type and origin, which have been assembled in some way to form a nonwoven fabric (a fiber layer, a fiber web) and bonded together in some way; this excludes the crossing or intertwining of yarns, as occurs in weaving, working, knitting. Films and paper are not considered nonwovens.

Thirdly: ‘Nappies, femcare’; absorbent products in various forms made of textile or textile-like fabrics, such as nonwovens, for the hygienic absorption of excretions.

Fourthly: “Textiles”; the term textiles comprises textile raw materials (natural fibers, chemical fibers) and non-textile raw materials, which are processed using various methods to form linear, sheet and three-dimensional structures, such as woven fabrics, worked fabrics, knitted fabrics, braided fabrics, sewn fabrics, nonwovens, floor coverings and felts and three-dimensional textile structures (body structures) such as textile tubings, stockings or textile semi-finished products for reinforced plastic components, wherein nonwovens are mentioned separately under ‘secondly’, although they are classified as textiles. Leather and imitation leather, for example, can also be examined.

Firstly, FIG. 2 shows the measuring device 100 according to the invention for determining softness as an individual parameter and other individual parameters of the sheet material in the open state.

As with the prior art, the open state means that the scraper element is not lying on a sample P. The open state is additionally illustrated in FIG. 2 in that a protective housing 108B is arranged and shown, which is arranged in a fixed position on a measuring head 108A of the measuring device 100, symbolized by a housing. In the open state, the protective housing 108B and the scraper element are located further above the measuring housing 108 than in FIGS. 3 to 6, which each show a closed state.

FIGS. 2 to 6 show the new structural features of the measuring device 100 according to the invention, which differ from the prior art, with the same reference signs as given below, wherein their functions in connection with the methods are explained in more detail in the following.

| 116A, 116B | first (lower) microphone and second (upper) |
|---|---|
| | microphone in combination |
| 106A | temperature generation and measuring device |
| | (thermofinger) |
| 148 | third motor |
| 138 | camera |
| 140 | vibration generator |
| 142 | temperature sensor |
| 144 | measuring device |

In other words, the measuring device 100 according to the invention has additional features that were not previously available, which are used to determine further individual parameters in order to be able to assess the properties of the sheet material in a user-friendly manner, wherein the term ‘sample P’ of the sheet material is used in the following again as representative of the materials mentioned.

Firstly, the individual parameters are defined in more detail, which in combination lead to a ‘haptic sensation’ for a person touching a sheet material, or are processed by a person to give a subjective impression of ‘haptic quality’.

The haptics are essentially determined by the following individual mechanical and chemical parameters, which, when combined, give the overall haptic sensation.

The property of ‘softness’ (‘micro-surface variations’) as an individual parameter of the sheet material:

The perceived softness is essentially determined by the micro-compressibility of the surface, the frictional force, the type of fiber and fiber structure (fiber stiffness) as well as the bonding and number of vertical fibers.

As explained above, the softness is determined using the known measuring device 100S and the associated known method.

The property of ‘roughness’ (‘macro-surface variations’) as an individual parameter of the sheet material:

The applicant has determined that the perceived roughness of sheet material is determined by the micro and/or macro texture of the surface. Such a micro and/or macro texture is present in the respective sheet material, or the micro and/or macro texture can be altered by certain processes, such as the ‘embossing process’ for example.

The applicant has determined that the softness of two samples P with the same optically measured roughness but different softness is interestingly assessed differently by a person touching the samples P by hand due to the different softness of the samples P with regard to roughness. It was found that the fiber structure influences the ‘roughness’ property in terms of the number and/or length and/or stiffness of the vertical fibers.

The property of ‘stiffness/elasticity’ as an individual parameter of the sheet material:

This property is determined by the fiber morphology of the sheet material, the fiber bonding of the sheet material, the material structure of the sheet material, and, in the case of tissue in particular, by the so-called refining process.

Mechanical cellulose pulps are produced either by grinding or milling wood fiber or wood waste, or the fiber material is released from wood chips or wood particles in a refining process.

P1/P2: Assessment of the properties ‘softness P1’ and ‘roughness P2’ of the sheet material as individual parameters of the sheet material.

### Method Step V-12:

The further developed measuring device 100 advantageously determines individual parameters that correspond to the subjective feeling of the human hand when touching sheet materials and which, in combination (as combination parameters), enable an objective assessment of the haptics of the sheet material, wherein the individual parameters can be examined by the measuring device 100 and thus represent an assessment aid.

FIG. 3 shows the measuring device 100 according to the invention as shown in FIG. 2, now in a closed operating state in an application state to illustrate the determination of the softness P1 and roughness P2 of the sheet material.

The basic principle of the method according to the invention is also that the specially shaped lamellae of the scraper element, i.e. the element 106 acting on the sample P, wherein the lamellae are fixed vertically in a rotating metal body (compare the illustration in FIG. 3), scrape the sample P to be measured with a defined pressing force F and at a defined rotational speed, which sample is fixed as a membrane on the measuring housing 108 as a measuring cell.

Depending on the selected rotational speed, the lamellae of the scraper element are set to vibrate in the frequency range of their natural resonance frequency (at approximately 6.5 kHz) by so-called ‘stick and slip processes’, the peaks of which depend on the softness P1 of the sample surface of the sheet material, which in turn is determined by the microcompressibility, the number of free/vertical fibers, the fiber morphology, the fiber type, hardwood fibers or softwood fibers.

As in the known procedure, prior to the measurement for determining the softness, a calibration is performed such that in a predeterminable range of the sound spectrum or frequency band of a specific softness a specific reference sound spectrum or a specific reference frequency bane is assigned, wherein a complex index objectively summarizing the softness of the sample P in the sense of various parameters is also assigned for this purpose.

In this measuring process, a peak is detected in the sound spectrum during the rotation of the scraper element, which is denoted ‘TS7’ for example. The corresponding height of the peak, this so-called peak ‘TS7’ obtained from the frequency analysis, is a measure of the softness P1 (‘micro-surface variations’) of the sample P, in particular of the sample surface of the sheet material being examined.

The sound spectrum for determining the peak ‘TS7’ is recorded by the lower first microphone 116A or by the upper second microphone 116B or by both microphones 116A, 116B in parallel.

A harder sample P, i.e., a less soft sample P, has a higher peak ‘TS7’ than a softer sample P. In the measuring device 100 according to the invention, this spectrum peak ‘TS7’ is preferably recorded by the upper first microphone 116A and processed in the evaluation and calibration unit 126. This procedure provides the softness P1 as an individual parameter.

This means that the evaluation of the height of the peak ‘TS7’ in comparison to the calibrated sound spectrum leads to the result that the peak ‘TS7’ correlates with the softness P1 of sample P, so that a softness index can now be calculated that differs from the calibrated complex softness index P1 and objectively correlates with the softness P1 of sample P.

This procedure provides the softness P1 as an individual parameter that can be used objectively as a haptic combination parameter.

At the same time, the micro and/or macro texture of sample P and the lamellae sliding over it cause sample P itself, which is fixed to the measuring housing 108 as a measuring cell like a membrane or eardrum, to vibrate vertically. Unlike the height of peak ‘TS7’ described above, the heights of these vibrations depend on the size of the unevenness, i.e., the roughness P2 or, in other words, the micro and macro texture of the sheet material.

It has been found that during the rotation of the scraper element, a peak with a certain height is also produced in the sound spectrum, now in a different frequency range during the vibration of the sample P, i.e., a further peak can be detected. This further peak is in the sound spectrum as a function of the rotational speed, for example between 0.4 and 1 kHz, wherein the detected value, i.e. this parameter value, represents the roughness P2 (‘macro-surface variations’) of the sample surface of the sheet material, as it is formed by the vibration of the sample P of the sheet material. This parameter is denoted ‘TS750’, for example.

The sound spectrum for determining the spectrum peak ‘TS750’ can be detected by the lower first microphone 116A or by the upper second microphone 116B or by both microphones 116A, 116B in parallel.

Accordingly, prior to the measurement for determining the roughness P2, a calibration is also carried such that a specific reference sound spectrum or a specific reference-frequency band is assigned in the prespecifiable range of the sound spectrum or the frequency band, wherein a complex index is also assigned for this purpose, which objectively summarizes the various parameters of the roughness P2 of the sample P.

This means that the evaluation of the height of the further peak ‘TS750’ in comparison to the calibrated sound spectrum leads to the result that the further peak ‘TS750’ correlates with the roughness P2 of the sample P, so that now a roughness index can now be calculated that differs from the calibrated complex roughness index P2 and objectively correlates with the roughness P2 of sample P.

This procedure makes roughness P2 (‘macro-surface-variations”) objectively available as a further individual parameter for use as a haptic combination parameter.

It has also been found that it is preferable to determine the spectrum peak ‘TS7’ using the upper second microphone 116B and to determine the spectrum peak “TS750” using the lower first microphone 116A, since the spectrum-peak ‘TS750’ generated by the vibration is more specific, i.e. can be detected more accurately, by the lower first microphone 116A because the sample P is fixed/stretched like a membrane or eardrum on the measuring housing 108 as a measuring cell.

P13/P23: Assessment of the properties ‘softness P1’ and ‘roughness P2’ of the sheet material as individual parameters that can be combined in terms of haptics, as a function of the porosity P3 of the sheet material.

### Method Step V-3:

With regard to the individual parameters of softness P1 and roughness P2, it was further found that the porosity P3 of the respective sample P of the sheet material influences the height of the spectrum peaks ‘TS7’ and ‘TS750’, wherein the influence of the porosity P3 or different porosities P3 of the samples P of the sheet material on the peak “TS750” is strong with regard to the roughness P2, while the influence of the porosity P3 or different porosities P3 on the peak ‘TS7’ is lower with regard to the softness P1 of the sheet material.

It was found that the measuring housing 108 (see FIGS. 3 to 6) forms a cavity as a measuring cell that is closed at the top by the membrane-like sample P. The sample P, which is attached to the measuring housing 108 as a measuring cell, acts as an amplifier of the air vibrations generated in the measuring cell due to its membrane vibrations.

It has been found that the greater the porosity P3 of the sample P, the lower the air compression in the measuring cell. This means that the height of the peaks ‘TS7/TS750’ of the air vibrations inside the measuring cell is lower, even though the height of the vibrations during the sample vibration of the sample P itself is the same.

It was thus recognized that the spectrum peaks ‘TS7’ and/or ‘TS750’ change depending on the porosity P3 of sample P and can be advantageously adjusted depending on the porosity P3, so that advantageously a more specific index of softness P1 and roughness can be determined than before, as the influence of porosity P3 is now taken into account, as explained in the following.

In other words, with a comparatively low or lower porosity P3 of the sample P, a strong or stronger air compression occurs in the measuring housing 108 as a measuring cell comparison to a sample P with a high or higher porosity, although the amplitudes of the vibrating sample P itself have the same magnitude.

FIG. 4 explains this further, showing the measuring device 100 according to the invention, as shown in FIG. 2, now in a closed operating state in a further application state.

As explained in detail the solution according to the invention consists in the fact that (cf. now FIG. 4) immediately before the previously explained procedure or after the explained procedure, a method step V-3 is carried out in which a vibration generator 140 arranged in the measuring head 108A, in which the drive 102 is also arranged, produces an imbalance which is transmitted to the scraper element via the axis 124. A constant vibration is produced, so that the constant vibration—but in this method step V-3 without rotation about the axis 124—is transmitted to the sample P.

As can be seen from the illustration in FIG. 4, in contrast to the illustration in FIG. 3, the vibration generator 140 now vibrates, and in FIG. 4, in this method step V-3, there is no rotation of the scraper element about the axis 124, so that in FIG. 4 the rotation arrow about the axis 124 shown in FIG. 3 is missing.

This means that a vibrating translational/pendulum movement of the scraper element is generated without rotation. The adjustment movement Δs with which the scraper element moves onto the sample P is set, and a predeterminable time period Δt is also set in which the scraper element acts on the sample P, wherein the predeterminable pressing force F is also set and controlled.

The height-adjustable scraper element is thus moved onto the flexible sample P with the specified pressing force F via the second motor 136 of the drive 102. The adjustable pressing force F is preferably 0.1 N, for example, whereby the adjustment distance Δs is specified via the force measurement of the force measuring device 118 and is then measured and monitored. For example, it is preferably provided that the vibration generator 140 is arranged on a carrier plate in the measuring head 108A, wherein the carrier plate also supports the first motor 134 and the second motor 136 of the drive 102.

During this measurement, which takes place in the specified time period Δt the membrane-like vibrations of the sample P again produce a noise with a specific sound spectrum, which forms a wave-like pattern, producing complex vibrations in the region of the sample P, in particular in the cavity which is closed off in a membrane-like manner by the probe P at the top and amplifies the vibrations.

The sound signal generated by this procedure is detected by the lower first microphone 116A and, based on this sound spectrum, mathematical correction functions are calculated by which the previously or subsequently measured spectrum-peaks ‘TS7’ and ‘TS750’ can be transformed or corrected.

The transformation means that a change in the individual parameters of softness P1 and/or roughness P2 takes place in such a way that the individual parameters of softness P13 and/or roughness P23 are indirectly corrected as a function of the porosity P3, as explained further in the following.

This method step V-3 is characterized in that, during the specified measurement period, the vibration generator 140 ensures that the scraper element vibrates, wherein the vibrations produced thereby are detected by means of vibration analysis to determine an additional sound spectrum or frequency band, which is detected by the first microphone 116A arranged inside the measuring housing which in its interior forms the enclosed measuring space underneath the sample, and processed in the evaluation and calibration unit 126 and evaluated in an evaluation device, in particular an external evaluation device, wherein a peak is generated and detected within the determined additional sound spectrum or frequency band as an individual parameter at a vibration frequency that can be specified by the vibration generator. The vibration of the sample P generated by the vibration generator—now rotation-free—forms a VRN peak with a specific height and a specific position in the additional sound spectrum or frequency band within the sound spectrum or frequency band, wherein the position and height of the VRN peak depend on the respective surface weight and the respective porosity of the sheet material being examined.

In an advantageous manner, the process is designed so that the specific peaks of the individual parameters of softness and/or roughness detected in the sound spectrum or frequency band are transformed by means of the mathematical correction function. This mathematical correction function transforms the specific peaks of the individual parameters of softness and/or roughness determined in the sound spectrum or frequency band as a function of the position generated by the rotation-free rotation, height of the VRN peak determined in the additional sound spectrum or frequency band and already explained, so that the dependence of the softness P1 and/or roughness P2 of the sheet material on the porosity is advantageously taken into account by the VRN peak characterizing the porosity P3.

This means that by combining the results of the previously explained method steps, an individual parameter of porosity-transformed softness and/or an individual parameter of porosity-transformed roughness can be determined in an advantageous manner. In other words, a correction function is applied within the calculation algorithm which transforms and corrects the influence of porosity with regard to the specific heights of the peaks of the individual parameters of softness P1 and/or roughness P2. This means that within the calculation algorithm for determining the softness P1 and/or roughness P2, the correction function is applied which takes into account the position and height of the VRN peak determined in method step V-3.

In summary, the individual parameters of softness P1 and roughness P2 are thus available, on the one hand, independently of the porosity P3 and, on the other hand, as the softness P13 and/or roughness P23 depending on the porosity P3, which is considered to be a significant advantage of the invention. In addition, the NRV peak described above is available as a parameter, which depends on the porosity of the sheet material and the surface weight.

P4: P4: Individual parameter ‘frictional force’ of the sheet material.

### Method Step V-4:

As explained above, the perceived softness, i.e., the haptics, is also determined by the surface friction of the sheet material, which is felt by humans when touching the sheet material.

According to the invention, the frictional force P4 of the sheet material is also determined as a measure by means of the measuring device 100.

The procedure essentially corresponds to method steps V-12 according to FIG. 3 for measuring softness, in which the sample P is set into rotation about the axis 124.

It is therefore similarly provided that the specially shaped lamellae of the scraper element, i.e. the element 106 acting on the sample P, with the lamellae being fixed vertically in a rotating metal body (cf. the illustration in FIG. 3), scrape the sample P to be measured with a defined pressing force F and at a defined rotational speed, which sample is fixed as a membrane on the measuring housing 108 as a measuring cell.

In order to determine the frictional force P4 produced during the rotational movement of the scraper element on the sample P, the first motor 134 is provided with a measuring device 144 (shown in FIGS. 2 to 6), and its measurement results correspond to the frictional force that occurs between the scraper element and the sample P.

It is provided that the initial frictional force P4.1, which occurs when the first motor 134 generating the rotation is started up, is derived from the measurement results, wherein the frictional force that is present when a constant rotational speed is reached is also derived from the measurement results and is assigned to the respective sheet material as the index frictional force P4.2.

It is clear that, in this procedure, the specially shaped lamellae of the scraper element are first applied to the sample P with the defined pressing force F and only then is the first motor 134 switched on and brought to a defined rotational speed on the sample P to be measured in order to derive the initial frictional force P4.1. The specially shaped lamellae of the scraper element scrape or rub the sample P, which is attached to the measuring cell.

In addition to the initial frictional force P4.1, it is also provided to derive the decreasing frictional force P4.3, which occurs over a longer, predeterminable time period, from the measurement results.

In other words, for each sheet material, a variable corresponding to the surface friction can be determined, which in relation to the initial frictional force P4.1 reflects the surface friction of a new, unused sheet material. The index frictional force P4.2 assigned to the respective sheet material reflects a specific surface friction attributable to the sheet material, which is independent of a new condition of the sheet material or an old condition of a worn sheet material. The measure of the derived decreasing frictional force P4.3 allows the wear rate of the sheet material and the maximum wear of the material occurring due to surface friction between the scraper element and sample P to be assessed for the respective sheet material. The defined pressing force F and the defined rotational speed of the scraper element acting on the sample P have a corresponding influence on the measurement results, wherein it may be provided that comparative measurements of a sheet material or of comparatively different sheet materials can be carried out at the same/different pressures and/or the same/different rotational speeds of the scraper element acting on the sample P.

The measuring device 144 for deriving the frictional force P4 can, for example, be a measurement of the current curve of the first motor 134, which is preferably in the form of a direct current motor. Of course, for example, the determined torque of the direct current motor can also be used to derive the frictional force P4.

According to the invention, the initial frictional force P4.1, the index frictional force P4.2 and the decreasing frictional force P4.3 from the index frictional force P4.2 are thus available as individual parameters, which is seen as a further significant advantage of the invention.

The frictional force P4 can be derived independently of the other method steps or measurements described.

P5: P4: Individual parameter ‘surface thermal conductivity’ of the sheet material.

### Method Step V-5:

FIG. 5 shows the measuring device 100 according to the invention as shown in FIG. 2 in a closed operating state that differs from FIGS. 2 to 4 in a further application state to explain the detection of a further individual parameter, namely the surface thermal conductivity P5.

Thermohaptics, as a branch of haptics, is essentially determined by the surface thermal conductivity P5, which in turn is influenced by the type of material, material density, material porosity and material roughness of the surface of the sheet material.

The less dense the surface, i.e., the more insulating air is present on the surface, the warmer the material feels, depending on the thermal conductivity of the material itself. The effect also occurs as a result of any coatings, i.e., the more lotion is applied, for example to tissues, the cooler the material feels, as the coating material at least partially displaces the heat-insulating air on the surface.

It is provided (cf. again FIG. 5) that at least one temperature generation and measuring device 106A is arranged in/on the measuring head 108A, by means of which the sheet material can be heated to a specific predetermined temperature, wherein the temperature generation and measuring device 106A, can simultaneously detect the temperature change of the sample P per unit of time in addition to the adjustable temperature. The temperature generation and measuring devices 106A are preferably NTC sensors, which are also referred to by experts as thermofingers. Such thermofingers advantageously fulfill the aforementioned functions.

It is provided that the thermofinger is placed on the sample P. It has been found that it is advantageous to use the existing mechanism, already been explained several times, but which now ensures that the thermofinger—and not the scraper element—rests on the sample P with a specific pressing force F.

It is now provided that it is no longer the specially shaped lamellae of the scraper element that come into contact with the sample P under a defined pressing force F, but rather that the thermofinger comes into contact with the sample P with the defined pressing force F.

It is further provided that, based on FIG. 2 (compare FIGS. 2 and 5), the measuring head 108A, i.e. the scraper element and the thermofinger together, are adjusted by a predeterminable adjustment distance Δs in a predetermined time period Δt in the direction of the sample P until the thermofinger presses on the sample P with its tip, wherein the predeterminable pressing force F is again controlled, which now no longer relates to the pressing of the scraper element, but to the pressing of the thermofinger on the sample P.

The thermofinger itself is also movable and connected to the measuring head 108A. It can be moved independently of the height-adjustable scraper element via a third motor 148 relative to the scraper element and also together with the scraper element when the measuring head 108A moves vertically upwards or downwards.

It is preferred that the thermofinger is firstly moved by a certain amount relative to the scraper element so that the tip protrudes beyond the lower side of the scraper element. The scraper element and the thermofinger are then moved together by a predetermined adjustment distance Δs in a predetermined time period Δt in the direction of the sample P, wherein the thermofinger is placed orthogonally on the flexible sample P with its tip with the predetermined pressing force F generated by the second motor 136 through its adjustment distance Δs. The prespecified pressing force F is preferably 0.01 N, for example.

It is also preferred that the thermofinger extends vertically through an opening in the scraper element so that, when in use, the thermofinger is located locally in the body (not shown) of the scraper element, so that, different from shown, the thermofinger forms a space-saving arrangement together with the scraper element.

In cases where the scraper element is set into rotation in this integrated arrangement during the described procedures, the thermofinger is moved completely out of the body of the scraper element by means of the third motor 148 and does not pass through the intended opening or openings of the scraper element. In other words, the thermofinger can be moved independently of the scraper element from an initial position to an operating position in which it finally rests on the sample P, since its tip protrudes from the underside of the scraper element by a predeterminable distance. The underside of the scraper element (see FIG. 5) does not touch the sample P when this method step V-5 of the invention is carried out. The scraper element, i.e., the measuring head 108A, is moved downwards together with the thermofinger as explained, wherein the thermofinger is extended a little further relative to the scraper element so that the thermofinger comes into contact with the sample P.

In the preferred embodiment, the thermofinger moves from the starting position to the operating position through at least one opening in the body of the scraper element. For this purpose, the scraper element is first rotated into the position in which the thermofinger passes precisely through the at least one opening and, in the operating position, protrudes with its tip on the underside of the scraper element by a predeterminable distance.

In this method step V-5, which takes place in the predeterminable period Δt the following procedure is carried out when the thermofinger is placed on the flexible sample P with its tip with the predetermined pressing force F. The thermofinger is switched on, wherein a predeterminable target temperature of the thermofinger is set. The thermofinger heats up and the temperature is measured continuously. The time period required to reach the target temperature from the initial temperature is hereby determined. The elapsed time period is a measure of the surface thermal conductivity P5. The greater the surface thermal conductivity P5 of the sheet material, the slower the target temperature is reached in the sensors and vice versa, as the heat supplied—similar to the heat supplied by a human hand—is dissipated into the sheet material.

In another embodiment, a specific heating current is applied to the sample P for a predeterminable time period by means of the thermofinger, after which the temperature curve is determined during this time.

In other words, the higher the thermal insulation or thermal insulation capacity P6 on the surface of the sheet material, the faster the temperature increases and/or the target temperature is reached earlier, since the heat supplied—similar to the heat supplied by a human hand—is not dissipated into the sheet material on the surface of the sheet material. It goes without saying that users who have high requirements for the thermal insulation of the sheet material are particularly interested in such objectively recorded measurement results with regard to surface thermal conductivity P5.

In terms of thermohaptics, sheet materials with higher surface thermal conductivity P5 feel cooler to the touch of a hand, as the heat supplied by the hand is quickly dissipated into the sheet material, while sheet materials with lower thermal conductivity on the surface, i.e. good thermal insulation P6, in particular on the surface when touched by a hand, feel warm because the heat on the surface of the sheet material supplied by the hand is only slowly dissipated into the sheet material.

An individual parameter is thus available as a measure of the surface thermal conductivity P5 of the sheet material, which is seen as a further key advantage of the invention.

The above description makes it clear that the individual parameter is the surface thermal conductivity P5, but the thermal insulation P6 as heat transfer through the sheet material is also of particular interest to the user.

P6: P4: Individual parameter ‘thermal insulation’ of the sheet material.

### Method Step V-6:

For users, the measuring device 100 according to the invention also provides an individual parameter that can be used to assess the ‘thermal insulation’ or thermal insulation capacity P6 of the sheet material. The metrological requirements correspond to the previous description of the thermofinger, which is placed on the flexible sample P with its tip with the specified pressing force F. The thermofinger is switched on to determine the individual parameter of ‘thermal insulation’ P6, wherein a predeterminable target temperature of the thermofinger is set. This may be the prespecified target temperature explained above, or a different target temperature is selected. The prespecified pressing force F is preferably 0.01 N, for example.

The thermofinger heats up in a predeterminable or predetermined time period and the temperature rising to the target temperature is measured continuously.

In contrast to the previous procedure, the time required to reach the target temperature from an initial temperature is not determined; instead, the temperature is increased up to the prespecified target temperature and maintained at the target temperature level once this target temperature has been reached.

As also illustrated in FIG. 5, the measuring device 100 according to the invention comprises not only the thermofinger, but also comprises, underneath the sample P, on the side of the sample P opposite the thermofinger, the temperature sensor 142, which is preferably an infrared (IR) sensor. The temperature sensor 142 detects the heat present in the region of the temperature sensor 142, wherein the temperature is recorded in a characteristic curve over time, which is used as a measure for assessing the thermal insulation of the sheet material. This means that the temperature sensor 142 detects a slow (very good, good thermal insulation properties) or fast (less good thermal insulation properties) increase in temperature over time, which is used to assess the quality of the thermal insulation of the sheet material.

For a sheet material with good thermal insulation properties, the gradient of the temperature increase of the characteristic curve is lower than for a sheet material with less good thermal insulation properties, since the heat is transferred more quickly to the temperature sensor 142 in a sheet material with less good thermal insulation properties than in a sheet material with good or very good thermal insulation properties.

In other words, this procedure provides the user with measured values and characteristic curves for the individual parameter ‘thermal insulation’ P6, which make it possible to objectively assess the thermal insulation properties, i.e., the heat transfer of the sheet material.

The thermal insulation P6 of the sheet material is thus also available as an individual parameter, which is considered to be another significant advantage of the invention.

The individual parameters described above, the measure of the surface thermal conductivity P5 of the sheet material on the surface and the measure of the thermal insulation P6 of the sheet material can be determined separately in the described procedures, or they can be combined into one procedure so that both measures are recorded together in one measurement process, meaning that the thermofinger only needs to be lowered into the described operating state once, which is advantageous.

P7: Individual parameter ‘lateral flexibility’ of the sheet material:

### Method Step V-7:

The procedure described in the following is illustrated in FIG. 6. FIG. 6 differs from FIGS. 3 and 4 in that there is no rotation via the first motor 134 as shown in FIG. 3 and no vibration via the vibration generator 140.

The structural arrangement for the procedure for determining the individual parameter of ‘lateral flexibility’ P7 of the sheet material is similar to the illustration of the measuring device 100 shown in FIG. 3, which in FIG. 3 is shown in the closed operating state (without vibration of the scraper element), but with rotation about the axis 124.

FIG. 6 shows the measuring device 100 in the closed operating state (without vibration) and, in contrast to FIG. 3, the scraper element does not rotate about the axis 124, to determine the individual parameter of ‘lateral flexibility’ P7.

The scraper element is thus moved vertically downwards onto the sample P without rotation (without starting the first motor 134 responsible for rotation) using the second motor 136, until a predetermined initial force, for example 100 mN, is detected by means of the force measuring device 118. A predeterminable end force, for example 600 mN, is detected when the scraper element has moved slowly and continuously into the flexible sample P. The speed at which the predeterminable end force is to be reached after reaching predetermined initial force is adjustable.

The displacement differential Δs covered by the measuring head 108A or the scraper element, which is pressed into the sample P, is recorded as a measure of the ‘lateral flexibility’ and made available to the user.

In other words, the user has at their disposal an objectively measured value for assessing the sheet material, i.e., a mechanical deformation value that corresponds to the ‘lateral flexibility’ P7 of the sheet material, wherein the viscoelastic and plastic deformation properties of the sheet material are disregarded in this procedure.

P8: P8: Individual parameter ‘recovery property’ of the sheet material:

### Method Step V-8:

When determining the ‘recovery property”, the scraper element is driven vertically downwards onto the sample P by the second motor 136, as previously explained for FIG. 6, without rotation (without starting up the first motor 134 responsible for rotation) until a predeterminable test force, for example 100 mN, is determined by means of the force measuring device 118. The speed at which the predetermined test force is to be achieved, starting from a rest position of the sample P is adjustable.

The determination of reaching the predetermined test force is carried out by means of the force measuring device 118, to which the test force exerted on the sample P is transmitted.

Once the test force has been reached and the sample P is now in a deflected deformation position, it is preferable to wait for a preset time period until the sample P, i.e. the elastic parts of the sheet material, have undergone reversible deformation and any plastic parts of the sheet material have undergone irreversible deformation.

If the sheet material consists exclusively of elastic parts that are reversibly deformed, the sample P returns to its original resting position after a relief time point at which the test force is withdrawn.

If the sheet material also has plastic parts that are irreversibly deformed, the sample P does not return to its original rest position after the relief time point at which the test force is withdrawn, but instead reaches a final deformation position that does not correspond to the original rest position.

Based on the predetermined test force that has moved the sample P from its rest position to the deflected deformation position, the time it takes for the sample P to return to its rest position is now determined.

The restoring force, i.e., the recovery force of the sheet material, moves back in the direction of the original rest position of the sample P.

The sample P is unloaded at the relief time point and the time period is determined until the sample P, starting from the relief time point, has reached its original rest position or the final deformation position at the later recorded end time point, which is determined by the fact that a set counterforce no longer changes at the later determined end time point.

The low counterforce acting on sample P after the unloading time point is exerted by driving the second motor 136, which moves slowly upwards as a result of the restoring force until the force measuring device 118 registers the amount of the specified counterforce exerted on sample P via the scraper element. The specified counterforce ensures that the scraper element is always in contact with the sample P.

This results in a restoring speed (distance/time unit) of the sample P between the relief time point and the later determined end time point, at which either the end deformation position or the original rest position of the sample P is reached.

The user thus has access to the ‘single-layer recovery property’ P8 of a single- or multi-layered sheet material as an individual parameter.

In the preferred embodiment of the invention, it is provided that the number of recovery-measurement cycles and/or the speed at which the sample P is deformed and/or the relief time point at which the test force is withdrawn, and/or the test force by which the recovery property is determined, can be set and predetermined.

P9: Individual parameter of ‘visual image documentation’.

### Method Step V-9:

As shown in FIGS. 2 to 6, the measuring device 100 according to the invention comprises the particularly high-resolution camera 138 for the image documentation of the sample P and additionally for the visual analysis of the surface of the sheet material.

The user thus also has access to a view of the surface or surface structure of the sheet material as an individual parameter, which can be used to assess sheet materials or compare sheet materials or to evaluate the aforementioned individual parameters for plausibility or associated dependencies between the individual parameters, which is also considered a significant advantage.

The measuring device 100 is also configured to carry out the method steps V-12, V-3, V-4, V-5, V-6, V-7, V-8 and V-9 of the method according to the invention.

For this purpose, the measuring device 100 comprises, in particular, a control device (computer with programmable logic controller), in which a computer-readable program algorithm for executing the method or the method steps and, if necessary, the required characteristics are stored. As explained, the control movements and evaluations according to the invention described above are carried out by the control device.

It is preferably provided that device-side control modules of a device-side control device (specifically a computer; programmable logic controller) are provided for controlling the motors 134, 136, 148, force measuring device 118, the displacement measuring device, the vibration generator 140, the temperature sensor 142 and the measuring device 114, the temperature generation and measuring device 106A and the internal evaluation and calibration unit 126. Preferably, an interface is provided by means of which the device-side control modules of the device-side control device communicate with an external evaluation device (computer), wherein, in particular, the evaluation and processing of the measurement results for the user are preferably carried out in the external evaluation device.

Based on the measuring device 100 of the first embodiment, a second embodiment of a measuring device 100′ is described in the following, which differs in some technical details from the measuring device 100 of the first embodiment. The measuring device of the second embodiment is shown under the reference sign 100′ with an apostrophe (′) in FIGS. 2A to 6A and is described with respect to the differences from the measuring device 100 of the first embodiment.

In the Figures:

FIG. 2A shows a measuring device according to the invention in a second embodiment in an open state;

FIG. 3A shows the measuring device according to the invention as shown in FIG. 2A, now in a closed operating state in an application state to explain the detection of individual parameters, namely the softness and roughness of the sheet material;

FIG. 4A shows the measuring device according to the invention as shown in FIG. 2A, now in a closed operating state in another application state for explaining the detection of a further variable characterizing the sheet material, from which the porosity-corrected softness and/or the porosity-corrected roughness of the sheet material can be derived as individual parameters;

FIG. 5A shows the measuring device according to the invention as shown in FIG. 2A in the closed operating state in another application state for explaining the detection of further individual parameters, namely a surface thermal conductivity and a thermal insulation of the sheet material;

FIG. 6A shows the measuring device according to the invention as shown in FIG. 2A, now in the closed operating state in another application state for explaining the detection of further individual parameters, namely the lateral flexibility of the sheet material and the resilience of the single-layer sheet material and the resilience of the multi-layer sheet material.

In FIGS. 2A to 6A, it is clear from the overview that the previous temperature generation and measuring device 106A, in combination with the temperature sensor 142, is referred to in the modified design variant of the second embodiment of the measuring device 100′ by the modified reference sign 106A′, 106.1A′, as explained in detail in the following.

Furthermore, the reference signs 140′, 150′ and 152′ in FIGS. 2A to 6A show that the arrangement and configuration of the previous vibration generator with the previous reference sign 140 has changed, as also explained in detail in the following.

In addition, FIGS. 2A to 6A show a further microphone 116C′, which is not provided in the first embodiment, and its function is explained in the following.

For a better overview, the structural features of the measuring device 100′ according to the invention of the second embodiment that are new compared to the prior art are given again with their reference signs, wherein the arrangements, configurations and functions of the features that have been changed compared to the first embodiment in connection with the procedures are explained in more detail in the following.

| 116A, 116B | first (lower) microphone and second (upper) |
|---|---|
| | microphone in combination and third microphone 116C′ |
| 106A′ | temperature generation and measuring device |
| | (thermofinger) with thermal imaging camera 106.1A′ |
| 148 | third motor |
| 138 | camera |
| 140′, 150′ | vibration generator with vibration element |
| 152′ | fourth motor |
| 142′ | temperature sensor as thermal imaging camera, |
| 144 | measuring device |

### Additional Microphone 116C′:

In contrast to the first embodiment of the measuring device 100, the measuring device 100′ in the second embodiment firstly has an additional microphone, in particular a third microphone 116C′. It also applies for this microphone 116C′ in that it represents, for example, any type of measuring device that is capable of receiving and registering sound spectra or frequency bands.

The third microphone 116C′ is preferably arranged inside the measuring housing 108A, wherein the measuring housing 108A has an opening in the region of the third microphone 116C′ so that noises from the environment of the measuring device 100′ can be recorded. The third microphone 116C′ is also connected to the evaluation and calibration unit 126.

The measuring device 100′ also has the two microphones 116A and 116B. When and how the microphones 116A and 116B are used in the first embodiment of the measuring device 100 in the method steps has already been explained in detail in connection with the first embodiment.

The parameters of softness P1, roughness P2 and softness P13 and/or roughness P23 are determined as a function of porosity P3 by means of one microphone 116A, 116B or by means of both microphones 116A, 116B.

Noises are always recorded, wherein the comparability of several measurements is assumed to be based on consistent environmental conditions, which are, however, slightly influenced by background noise. However, in practice, this background noise is sometimes unavoidable, so that the measuring device 100′ is now equipped with an additional microphone 116C′, which is switched on in parallel when at least one of the microphones 116A, 116B is used to determine the background noise in parallel. The evaluation and calibration unit 126 further comprises, within the scope of the invention, the computer-readable program algorithm for executing the method or the possible method steps, which is configured in such a way that it corrects the noises recorded by the microphone(s) 116A, 116B in such a way that the background noise detected by the third microphone 116C′ is corrected mathematically in the noise recorded by the microphone(s) 116A, 116B. In other words, the measured background noise is ‘calculated out’ of the measurement results of microphone 116A or 116B or microphones 116A and 116B. As a result, the parameters of softness P1, roughness P2 and softness P13 and/or roughness P23 can be better compared with each other as a function of the porosity P3 of several measurements, since the effect that several measurements may produce deviating measurement results due to any background noise is eliminated.

### Modified Arrangement and Configuration of the Vibration Generator 140′:

The measuring device 100′ in the second embodiment also has a modified arrangement for generating vibrations in the sheet material of the sample P.

In the configuration of the first embodiment according to measuring device 100, the at least one vibration generator 140 is arranged in the measuring head 108A, in which the drive 102 is also arranged. In the previous method step V-3, the vibration generator 140 generates an imbalance in the measuring head 108A, in which the drive 102 is also located, which is transmitted to the scraper element via the axis 124. A constant vibration is generated, wherein the constant vibration is transmitted to the sample P in method step V-3 without rotation about the axis 124.

In method step V-3, the vibration generator 140 ensured that the scraper element, which did not rotate in this method step V-3, and thus the sample P, vibrated during the specified measurement period.

It has been found that it is advantageous that the vibration of the sample P can be adjusted more conveniently by a separate vibration generator 140′ that is independent of the scraper element.

In the modified variant of the measuring device 100′, it is now provided that the at least one vibration generator 140′ is arranged underneath the measuring head 108A on the measuring housing 108 independently of the drive 102 and the scraper element.

The vibration generator 140′ now comprises a vibration element 150′, which is denoted and configured as a vibration stamp. The vibration stamp forms a vibration transmitting surface at one end when it strikes the sample P of the sheet material orthogonally to the surface plane of the clamped sheet material, as shown in FIG. 4A and explained further in the following.

In the modified variant, the vibration generator 140′ is equipped with a fourth motor 152′. The fourth motor 152′ is used to place the vibration element 150′ on the stretched sheet material and thus on the sample P.

Method step V-3 has already been discussed. The method for determining individual parameters of sheet materials, such as flat hygiene paper and flat, textile-like or textile materials by means of the measuring device 100, remains fundamentally unchanged according to the third method step V-3, however, it is no longer the scraper element that is moved in a translational manner as a translational element 106 relative to the positionally fixed, at least single-layer sample P of sheet material with the predeterminable pressing force F onto the sample P, but rather the vibration stamp of the vibrations generator 140′.

The vibration stamp 150′ makes contact with the sample P, causing a noise to be generated during the predetermined measurement period after the vibration generator 140′ (cf. motor for reference numeral 140′) has been switched on, which is then received and recorded by the first microphone 116A.

During the predetermined measurement period, the vibration generator 140′ ensures that the sample P vibrates due to the applied vibration stamp, wherein the vibrations generated on the sample side are in turn detected, as before, by means of an additional vibration analysis to determine an additional sound spectrum or frequency band by the first microphone 116A arranged inside the measuring housing 108A, which forms in its interior the enclosed measuring chamber 110 underneath the sample P.

The specific peak determined in this way, as before, and its position in the additional sound spectrum or frequency band is a variable the measure of which characterizes a porosity P3 of the sheet material, wherein the variable is also determined using a calculation algorithm. According to the previous description, as before, method step V-3 is carried out before or after method step V-12, wherein the specific peaks of the individual parameters of softness P1 and/or roughness P2 determined in the sound spectrum or frequency band are corrected and transformed by means of a mathematical correction function that contains the measure of the variable characterizing the porosity P3 of the sheet material, so that, by combining the results of method step V-12 and method step V-3 as a function of the measure of the variable characterizing the porosity P3 of the sheet material, an individual parameter of the porosity-corrected softness P13 and/or an individual parameter of the porosity-corrected roughness P23 is/are obtained.

The fourth motor 152′ is used, with a partly lowered measuring head 108A (cf. FIG. 4A with FIG. 2A ‘not lowered’ and FIG. 3A ‘fully lowered’), to bridge the path of the vibration stamp to the sample P when the vibration stamp is to be placed on and then removed again from the sample P.

### Modified Embodiment of the Temperature Generation and Measuring Device 106A′ and the Temperature Sensor 142′

The temperature generation and measuring device 106A′ and the temperature sensor 142′ of the measuring device 100 are used in method steps V-5 and V-6, wherein the previously described method step V-5 is modified and is thus denoted V-5′ with an apostrophe (′).

In method step V-5′, a specific surface thermal conductivity P5 is derived as a further individual parameter depending on the measurement results and assigned to the sample P.

In the unchanged method step V-6, a specific thermal insulation capacity P6 is derived as a further individual parameter as a function of the measurement results in this method step and assigned to the sample P.

The modified embodiment continues to be a temperature generation and measuring device 106A′ and a temperature sensor 142′, which can thus be denoted as such, wherein a different embodiment is proposed, which has also proven to be advantageous.

The temperature generation and measuring device 106A′ is again denoted as a thermofinger, as in the previous embodiment.

The temperature generation and measuring device 106A′ also comprises an NTC sensor as a temperature generation device for generating heat. The NTC-sensor of the thermofinger brings about a specific temperature of the sample P at a local point of the sample P by supplying heat.

It is intended that (cf. FIG. 5) the thermofinger is placed on the sample P as before. As before, the existing and repeatedly explained mechanism (drive via the first motor 134) is used for this purpose, wherein it is further ensured that the thermofinger (cf. FIG. 5A) is placed or positioned on the sample P with a specific pressing force F, independently of the scraper element.

As before, the thermofinger is movable and connected to the measuring head 108A. The thermofinger can be moved firstly by the second motor 136 together with the scraper element, when the measuring head 108A moves vertically up or down, and secondly by the third motor 148, independently of the height-adjustable scraper element relative to the scraper element or the measuring head 108A.

### Modified Method Step V5′

It is preferable that the thermofinger (cf. FIG. 5A) is firstly moved together with the scraper element by a certain amount and then independently of the scraper element relative to the scraper element, so that the tip of the thermofinger protrudes beyond the underside of the scraper element.

Subsequently, the scraper element and the thermofinger are moved together by a predeterminable adjustment distance Δs in a specified time period Δt in the direction of the sample P, wherein the thermofinger is placed orthogonally on the flexible sample P with its tip with the predetermined pressing force F, which the second motor 136 generates according to its adjustment distance Δs and the contact of the tip with the sample P.

Before making contact, the thermofinger is heated to a specific predetermined temperature in a predetermined measurement period.

In the modified embodiment, the heat is only transferred to the sample P during a short, predetermined application period of the tip (e.g., tip of the thermofinger approximately 30° application period, e.g., 1 to 10 s).

At the end of the predetermined application period, the thermofinger or its tip is immediately moved away from the sample P by means of the third motor 148, wherein this method step V-5′ differs from method step V-5 of the measuring device 100 of the first embodiment.

The individual parameter, the surface thermal conductivity P5, is now derived in a different technical manner.

In the modified embodiment, the temperature generation and measuring device 106A′ comprises a thermal imaging camera 106.1A′, which advantageously determines, without contact, a temperature curve of the cooling, previously heated location of the sample P.

The non-contact nature of the thermofinger on the sample side means that, when recording the temperature curve, the influence of the tip of the thermofinger during the determination of the surface thermal conductivity is no longer present, as was previously the case.

Of course, the thermofinger is returned to its starting position after the temperature curve has been recorded.

As explained in the first embodiment of the measuring device 100, the measuring device 100′ thus provides the individual parameter of surface thermal conductivity P5 of the sheet material, which is seen as a further significant advantage of the invention.

As already explained, in addition to the individual parameter of surface thermal conductivity P5, for the user the thermal insulation P6 of the sheet material, i.e., the heat transfer through the sheet material of the sample P, is also of great interest.

In the measuring device 100′ of the second embodiment, the temperature sensor 142′ (cf. FIG. 6A) is also advantageously a thermal imaging camera. The thermal imaging camera detects the heat present in the region of the temperature sensor 142′, wherein the temperature is recorded in a characteristic curve over time, which in turn is used as a measure for assessing the thermal insulation of the respective sample P of the sheet material. This means that the temperature sensor 142′ detects a slow (very good, good thermal insulation properties) or fast (less good thermal insulation properties) increase in temperature over time, which is used to assess the quality of the thermal insulation of the sheet material. In the case of a sheet material with good thermal insulation properties, the gradient of the temperature increase of the characteristic curve is lower than in the case of a sheet material with less good thermal insulation properties, since the heat is transferred more quickly to the temperature sensor 142′ in the case of a sheet material with less good thermal insulation properties than in the case of a sheet material with good or very good thermal insulation properties.

In other words, this procedure, which is essentially unchanged from method step V-6 of the first embodiment, also provides the user of the measuring device 100′ with measurement values and characteristic curves for the individual parameter of ‘thermal insulation’ P6, which make it possible to objectively assess the thermal insulation properties, i.e. the respective heat transfer though the sheet materials being examined.

Like measuring device 100, measuring device 100′ of the second embodiment is configured to perform method steps V-12, V-3, V-4, V-6, V-7, V-8 and V-9 of the method according to the invention, wherein in the measuring device 100′ only method step V5′ differs from method step V5 of the measuring device 100 according to the first embodiment. In this respect, the method steps V-12, V-3, V-4, V5′, V-6, V-7, V-8 and V-9 can be carried out with the measuring device 100′ of the second embodiment.

For this purpose, the measuring device 100′ also comprises, in particular, a control device (computer with programmable logic controller), in which a computer-readable program algorithm for executing the method or the method steps and, if necessary, the required characteristic maps are stored. As explained, the control movements and evaluations according to the invention described above are carried out by the control device.

It is also provided preferably that there are device-side control modules of a device-side control device (specifically a computer; programmable logic controller) for controlling the motors 134, 136, 148, 152′, the force measuring device 118, the displacement measuring device, the vibration generator 140′, the temperature sensor 142′ and the measuring device 114, the temperature generation and measuring device 106A′, 106.1A′ and the microphones 116A, 116B and 116C′, as well as the other components required to carry out the method and explained in the description, and the internal evaluation and calibration unit 126. Preferably, the interface is configured to enable the device-side control modules of the device-side control device to communicate with an external evaluation device (computer), wherein the evaluation and processing of the measurement results for the user is preferably carried out in the external evaluation device.

### REFERENCE SIGNS

100S measuring device (prior art, FIG. 1)
100 measuring device (invention, FIGS. 2 to 6) in a first embodiment
100′ measuring device (invention, FIGS. 2A to 6A) in a second embodiment
102 drive
104 transmission means (drive shaft)
106 element (scraper element)
106A temperature generation and measuring device
106A′ temperature generation and measuring device
106.1A′ thermal imaging camera
108 measuring housing
108A measuring head
108B protective housing underneath the measuring head
110 measuring chamber (sound field)
112 sealing element (rubber lip)
114 holding element
116A first (lower) microphone
116B second (upper) microphone
116C′ third (lower) microphone
118 force measuring device
120 base
122 sample plane
124 axis
126 evaluation and calibration unit
128 temperature sensor (T) (temperature)
130 humidity sensor (ω) (relative humidity)
132 displacement measuring device
134 first motor
136 second motor
138 camera
140 vibration generator
140′ vibration generator
142 temperature sensor
142′ temperature sensor
144 measuring device
148 third motor
150′ vibration element
152′ fourth motor
F penetration force
P sample
p sound pressure
f frequency
I sound intensity
L sound pressure level
K softness index
Δt measuring period
Δs displacement differential
T temperature
ω humidity

1. A measuring device (100, 100′) for determining individual parameters of sheet materials, such as flat hygiene paper and flat, textile-like or textile materials, wherein the measuring device (100, 100′) comprises a measuring head (108A) and a measuring housing (108), wherein a scraper element (106) is arranged on the measuring head (108A), which element is movably arranged relative to a positionally fixed, at least single-layer sample (P) of the sheet material arranged on the measuring housing (108) and is arranged to be adjustable relative to the sample (P) according to a predetermined pressing force (F) acting on the sample (P), characterized in that a first microphone (116A) is arranged inside the measuring housing (108), which forms in its interior an enclosed measuring chamber (110) underneath the sample (P), and a second microphone (116B) is arranged outside the measuring housing (108), which together or independently of each other record noises produced by the relative movement of the scraper element (106) acting on the sample (P).

2. The measuring device (100) according to claim 1, characterized in that at least one vibration generator (140) is arranged in the measuring head (108A), which vibration generator transmits a vibration to the scraper element (106) and from the scraper element (106) to the sample (P).

3. The measuring device (100′) according to claim 1, characterized in that at least one vibration generator (140′) is arranged on the measuring head (108A), which vibration generator transmits a vibration directly to the sample (P) via a vibration element (150′).

4. The measuring device (100, 100′) according to claim 1, characterized in that the measuring head (108A) comprises a first motor (134), a second motor (136) and a third motor (148), wherein
the first motor (134) causes a rotational movement of the scraper element (106) relative to a sample surface of the positionally fixed, at least single-layer sample (P),
while the second motor (136) causes an orthogonal translational movement or a substantially orthogonal translational pendulum movement of the scraper element (106) relative to the sample surface of the positionally fixed, at least single-layer sample (P),
while the third motor (148) causes an orthogonal displacement of a temperature generation and measuring device (106A, 106A′) movably arranged on the measuring head (108A) relative to the sample surface of the positionally fixed, at least single-layer sample (P).

5. The measuring device (100′) according to claim 3, characterized in that the measuring head (108A) comprises a fourth motor (152′), wherein the fourth motor (152′) causes an adjustment movement of the vibration element (150′) of the vibration generator (140′) arranged on the measuring head (108A).

6. The measuring device (100, 100′) according to claim 4, characterized in that the first motor (134) is connected to a measuring device (144) which, during the rotational movement of the scraper element (106) generated by the first motor (134), determines and records a current curve and/or a torque and/or a power consumption of the first motor (134).

7. The measuring device (100, 100′) according to claim 1, characterized in that at least one temperature sensor (142, 142′) is arranged in the measuring chamber (110) at a predeterminable distance from the positionally fixed, at least single-layer sample (P).

8. The measuring device (100′) according to claim 1, characterized in that at least one further microphone (116C′) is arranged in the measuring chamber (110).

9. The measuring device (100, 100′) according to claim 1, characterized in that at least one camera (138) is arranged in or on the measuring head (108A) for image documentation of the sample (P) and additionally for visual analysis of the surface of the sheet material of the sample (P).

10. A method for determining individual parameters of sheet materials, such as flat hygiene paper and flat, textile-like or textile materials, by means of the measuring device (100, 100′), wherein in one method step (V-12) a movable scraper element (106) is moved in translation relative to a positionally fixed, at least single-layer sample (P) of the sheet material with a predeterminable pressing force (F) onto the sample (P), while simultaneously rotating said scraper element (106), and makes contact with and acts on said sample (P), whereby a noise is produced, received and recorded in a predetermined measuring period, characterized in that the vibrations generated are detected by means of a vibration analysis to determine a sound spectrum or a frequency band by a first microphone (116A) arranged inside a measuring housing (108), which forms in its interior an enclosed measuring chamber (110) underneath the sample (P), and by a second microphone (116B) arranged outside the measuring housing (108), wherein a specific softness (P1) and a specific roughness (P2) are assigned to the sheet material as individual parameters of the sample (P) of the sheet material by applying a calculation algorithm based on specific peaks determined in the sound spectrum or frequency band.

11. A method for determining individual parameters of sheet materials, such as flat hygiene paper and flat, textile-like or textile materials, by means of the measuring device (100, 100′), wherein in one method step (V-3) the scraper element (106) or a movable vibration element (150′) is moved in translation relative to a positionally fixed, at least single-layer sample (P) of the sheet material with a predeterminable pressing force (F) onto the sample (P) and makes contact with and acts on said sample (P), whereby a noise is produced, received and recorded in a predetermined measuring period, characterized in that, in the predetermined measuring period, a vibration generator (140) causes the scraper element (106) or the vibration element (150′) to vibrate, wherein the vibrations generated thereby on the sample side are detected by means of an additional vibration analysis to determine an additional sound spectrum or a frequency band by the first microphone arranged inside the measuring housing (108), which forms in its interior the enclosed measuring chamber (110) underneath the sample (P), wherein a detected specific peak and its position in the additional sound spectrum or frequency band is a variable the measure of which characterizes a porosity (P3) of the sheet material, wherein the variable is also determined using a calculation algorithm.

12. The method according to claims 10 and 11, characterized in that the method step (V-3) according to claim 11 is carried out before or after the method step (V-12) according to claim 10, wherein the specific peaks of the individual parameters of softness (P1) and/or roughness (P2) determined in the sound spectrum or frequency band are corrected and transformed by means of a mathematical correction function which contains the measure of the variable characterizing the porosity (P3) of the sheet material, so that, by combining the results of method step (V-12) and method step (V-3) as a function of the measure of the variable characterizing the porosity (P3) of the sheet material, an individual parameter of the porosity-corrected softness (P13) and/or an individual parameter of the porosity-corrected roughness (P23) is/are obtained.

13. A method for determining individual parameters of sheet materials, such as flat hygiene paper and flat, textile-like or textile materials, by means of the measuring device (100, 100′), wherein in one method step (V-4) a movable scraper element (106) is moved in translation relative to a positionally fixed, at least single-layer sample (P) of the sheet material with a predeterminable pressing force (F) onto the sample (P), while simultaneously rotating the scraper element (106), and makes contact with and acts on said sample (P), characterized in that, during the generated rotational movement of the scraper element (106), in a predetermined measuring period, a current curve and/or a torque and/or a power consumption of a first motor (134) that causes the rotational movement is/are determined and recorded, so that, depending on the respective measurement result, a specific frictional force (P4) is indirectly derived as an individual parameter and assigned to the sample (P), which frictional force is generated by the contact between the sample (P) and the scraper element (106) during the rotational movement.

14. A method for determining individual parameters of sheet materials, such as flat hygiene paper and flat, textile-like or textile materials, by means of the measuring device (100), characterized in that, in one method step (V-5), a movable temperature generation and measuring device (106) is moved in translation relative to a positionally fixed, at least single-layer sample (P) of the sheet material with a predeterminable pressing force (F) onto the sample (P) and makes contact with and acts on a surface of said sample (P), wherein the sheet material is either heated to a specific predetermined temperature by means of the temperature generation and measuring device (106) in a predetermined measuring period and the temperature change of the sample (P) per unit of time is simultaneously recorded in the predetermined measuring period, or a specific heating current is applied to the temperature generation and measuring device (106A) for a predeterminable time, after which the temperature curve is determined during this time, so that, depending on the measurement results, a specific surface thermal conductivity (P5) is derived as an individual parameter and assigned to the sample (P).

15. A method for determining individual parameters of sheet materials, such as flat hygiene paper and flat, textile-like or textile materials, by means of the measuring device (100′), characterized in that, in one method step (V-5′), a specific heating current is applied to a movable temperature generation and measuring device (106A′) for a predeterminable time and the temperature generation and measuring device is then is moved in translation relative to a positionally fixed, at least single-layer sample (P) of the sheet material with a predeterminable pressing force (F) onto the sample (P) and makes contact with and acts on a surface of said sample (P) for a predeterminable time period, wherein the sheet material is heated to a specific predetermined temperature by means of the temperature generation and measuring device (106A′) in a predetermined measuring period and the temperature generation and measuring device (106A′) is then removed from the sample (P) so that contact is broken, after which a thermal imaging camera (106.1A′) is used to determine a temperature curve of the sheet material of the sample (P) so that, depending on the measurement results, a specific surface thermal conductivity (P5) is derived as an individual parameter and assigned to the sample (P).

16. A method for determining individual parameters of sheet materials, such as flat hygiene paper and flat, textile-like or textile materials, by means of the measuring device (100, 100′), characterized in that, in one method step (V-6), a movable temperature generation and measuring device (106A, 106A′) is moved in translation relative to a positionally fixed, at least single-layer sample (P) of the sheet material with a predeterminable pressing force (F) onto the sample (P) and makes contact with and acts on a surface of said sample (P), wherein the sheet material is heated to a specific predetermined temperature by means of the temperature generation and measuring device (106A, 106A′) in a predetermined measurement period, wherein a temperature sensor (142, 142′) is arranged underneath the sample (P), on a side of the sample (P) opposite the temperature generation and measuring device (106A, 16A′), at a predetermined distance from the sample (P), which temperature sensor detects a temperature, wherein the detected temperature is recorded and registered in a characteristic curve over time, so that, depending on the measurement results, a specific thermal insulation capacity (P6) is derived as an individual parameter and assigned to the sample (P).

17. A method for determining individual parameters of sheet materials, such as flat hygiene paper and flat, textile-like or textile materials, by means of the measuring device (100, 100′), wherein in one method step (V-7) a movable scraper element (106) is moved in translation relative to a positionally fixed, at least single-layer sample (P) of the sheet material with a predeterminable pressing force (F) onto the sample (P) and makes contact with and acts on said sample (P), characterized in that, in a predetermined measurement period, a displacement differential (Δs) covered by the scraper element (106) when pressing the scraper element (106) onto the sample (P) is determined and recorded, so that, depending on the measurement results, a specific lateral flexibility (P7) is derived as an individual parameter and assigned to the sample (P).

18. A method for determining individual parameters of sheet materials, such as flat hygiene paper and flat, textile-like or textile materials, by means of the measuring device (100, 100′), wherein in one method step (V-8) a movable scraper element (106) is moved in translation relative to a positionally fixed sample (P) of the sheet material with a predeterminable pressing force (F) onto the sample (P) and makes contact with and acts on said sample (P), characterized in that the pressing force (F) is a predetermined test force which moves the sample (P) from its rest position into a deflected deformation position in at least one measurement cycle, wherein, in the at least one measurement cycle, a speed at which the predetermined test force is to be reached is predetermined, wherein the sample (P) returns to its final deformation position or its original rest position at a predetermined relief time point in the at least one measurement cycle, at which relief time point the test force is withdrawn, wherein, in a predetermined measurement period of the measurement cycle, a displacement differential (Δs) covered by the sample (P) and a time period in which the sample (P) returns from the deflected deformation position to the final deformation position or the original rest position are determined and recorded, so that, depending on the measurement results, a resilience (P8) of the sample (P) is derived as an individual parameter and assigned to the sample (P), wherein furthermore a number of measurement cycles and/or the speed at which the sample (P) is deformed and/or the relief time point at which the test force is withdrawn and/or the test force, at which the resilience is determined, can be predetermined.

19. A method for determining individual parameters of sheet materials, such as flat hygiene paper and flat, textile-like or textile materials, by means of the measuring device (100, 100′), characterized in that, in one method step (V-9), the surface structure of the sample (P) is photographed by a high-resolution camera (138), so that visual image documentation (P9) of the sample (P) is available as an individual parameter, wherein further individual parameters are verified as a function of the photographic reproduction of the surface structure.
